(19) United States
(12) Patent Application Publication
(10) Pub. No.: US 2026/0283833 A1
(43) Pub. Date: Sep. 24, 2026

(54) RESTRAINT SYSTEMS, DEVICES AND METHODS

(71) Applicant: Regents of the University of Minnesota, Minneapolis, MN (US)

(72) Inventor: Ann Seppelt, Sauk Rapids, MN (US)

(73) Assignee: Regents of the University of Minnesota, Minneapolis, MN (US)

(21) Appl. No.: 19/474,363

(22) PCT Filed: Apr. 12, 2024
(86) PCT No.: PCT/US2024/024482

§ 371 Date: Oct. 10, 2025

Related U.S. Application Data
(60) Provisional application No. 63/458,725, filed on Apr. 12, 2023.
(60) Provisional application No. 63/599,705, filed on Nov. 16, 2023.

(51) Int. Cl. A61F 5/37

(52) U.S. Cl. CPC A61F 5/3776; A61F 5/3707

(57) ABSTRACT

Restraining systems and devices for deployment or placement over a prone or supine subject's upper torso and/or limbs. Upper torso restraining devices (20) have a shell-like shape, defining an interior region sized and shaped to receive an upper torso of a human adult subject. The upper torso restraining device is sized and shaped to pin or contain the subject relative to a supporting surface and has a sufficiently robust construction such that a user can safely place her or his weight on a breastplate surface to prevent the subject from moving. Where appropriate, the restraining device can be strapped or secured to the supporting surface. Limb restraining devices are configured to be applied about or around a joint of a subject. Restraining systems can include the upper torso restraining device and one or more of the limb restraining devices.

### BACKGROUND

The present disclosure relates to devices for temporarily, and safely, restraining an individual. More particularly, it relates to devices for use during the often dangerous and violent take-down phase of subduing, containing and immobilizing a violent/agitated individual.

The need to quickly restrain and/or immobilize an individual can arise in a number of situations. For example, law enforcement officers, healthcare providers, etc., often encounter scenarios where a suspect or patient needs to be restrained as rapidly as possible to reduce both the potential for harm to the person to be restrained as well as to the officer or medical personnel. In many instances, a typical course of action is to force the suspect/patient to the ground or other firm surface and immobilize them in either the prone or supine position. One conventional technique used during what is known as the “takedown” phase to contain a violent/agitated individual entails exerting human body weight on the chest or neck once the individual is prone or supine. This approach can be highly dangerous to both personnel and the individual as adverse events such as suffocation, injury, permanent disability, and death have resulted historically. Additionally or alternatively, the ankles and wrists of the suspect/patent can be grasped and held by hand in hopes of avoiding injury to the major joints, but this can be exceedingly difficult and time-consuming to accomplish. Buckled restraints are available, but are slow to apply and often impossible to use with a thrashing subject until containment has been achieved.

### SUMMARY

The inventor of the present disclosure has recognized a need to address one or more of the above-mentioned problems.

Some aspects of the present disclosure are directed to systems, devices and methods for quickly and safely constraining a subject (e.g., patient). Some embodiments include a lightweight, upper torso restraining device adapted to be quickly deployed and arranged over a subject, holding the subject to a floor or other flat surface. The upper torso restraining device shields a user from kicks, punches, and spittle by the subject; contains the torso, hips, and/or limbs against a flat surface, allows visualization of the patient face and neck, and minimizes physical contact between user and subject. Once arranged over the subject, a user can place her or his weight on a surface of the upper torso restraining device, with a structure of the device protecting the subject from possible injury, impeded breathing, etc. Other embodiments include one or more limb restraining devices configured to be quickly deployed over the elbow or knee of the subject, serving to safely immobilize the subject's limb. In some examples, the limb restraining devices can include handles, straps, and other optional features.

### BRIEF DESCRIPTION OF THE DRAWINGS

FIG. 1A is a simplified perspective view of an upper torso restraining device in accordance with principles of the present disclosure arranged over a subject;

FIG. 1B is an end view of the upper torso restraining device of FIG. 1A;

FIG. 1C is a top view of the upper torso restraining device of FIG. 1A;

FIG. 2 is a simplified top view an upper torso restraining device in accordance with principles of the present disclosure arranged over a subject;

FIG. 3 is a simplified end perspective view of another upper torso restraining device in accordance with principles of the present disclosure;

FIG. 4 is a simplified end view of another upper torso restraining device in accordance with principles of the present disclosure arranged over a subject;

FIG. 5 is a simplified end view of another upper torso restraining device in accordance with principles of the present disclosure arranged over a subject;

FIG. 6 is a simplified end view of another upper torso restraining device in accordance with principles of the present disclosure arranged over a subject;

FIG. 7 is a perspective view of another upper torso restraining device in accordance with principles of the present disclosure;

FIGS. 8A and 8B illustrate a storage state of the restraining device of FIG. 7;

FIG. 9 is a simplified, perspective view of another upper torso retraining device in accordance with principles of the present disclosure;

FIG. 10 illustrates use of the restraining device of FIG. 9 in restraining a subject;

FIG. 11 is a simplified, perspective view of another upper torso retraining device in accordance with principles of the present disclosure;

FIG. 12 is a simplified top view of several restraining devices in accordance with principles of the present disclosure applied to a subject;

FIG. 13 is a simplified top view of several limb restraining devices in accordance with principles of the present disclosure applied to a subject;

FIG. 14 is a simplified top view of limb restraining devices in accordance with principles of the present disclosure applied to a subject;

FIG, 15 is a simplified top view of the arrangement of FIG. 14 during use with the subject on an auxiliary support;

FIG. 16A is a side perspective view of a limb restraining device in accordance with principles of the present disclosure;

FIG. 16B is a transverse cross-sectional view of the limb restraining device of FIG. 16A;

FIG. 17A is an enlarged cross-sectional view of a portion of a core useful with the limb restraining device of FIG. 16A;

FIGS. 18A and 18B are cross-sectional views illustrating transitioning of the limb restraining device of FIG. 16A;

FIG. 19 is a side perspective view of a limb restraining device in accordance with principles of the present disclosure;

FIG. 20 is a side perspective view of a limb restraining device in accordance with principles of the present disclosure;

FIG. 21 is a simplified end view of two of the limb restraining devices of FIG. 20 coupled to one another;

FIG. 22A is a side perspective view of a limb restraining device in accordance with principles of the present disclosure and in an open state;

FIG. 22B is an end view of the limb restraining device of FIG. 22A in a closed state; and

FIG. 23 is a simplified top view of a restraining system in accordance with principles of the present disclosure applied to a subject.

### DETAILED DESCRIPTION

The present disclosure relates to restraint systems, devices and methods for use in rapidly, and safely, restraining and/or immobilizing an individual. In some embodiments, the systems and devices of the present disclosure are configured for deployment or placement over a prone or supine subject's upper torso or other anatomy, serving to constrain the subject while protecting the subject from being harmed or impeding the subject's ability to freely breathe. In other embodiments, the systems and devices of the present disclosure are configured for deployment or placement over one or more limbs or joints of a subject. In yet other embodiments, the systems of the present disclosure can include an upper torso restraining device and limb restraining devices. In some embodiments, the restraint systems and devices of the present disclosure, or components thereof, are lightweight while also very strong for reduced risk of being used to cause injury by a combative person or to personnel using/applying the restraint. The systems of the present disclosure can sometimes be referred to as a Safeguard Shield and Containment System (SSCS) that optionally consist of a Safeguard Containment Device (SCD) and four Safeguard Limb Devices (SLD).

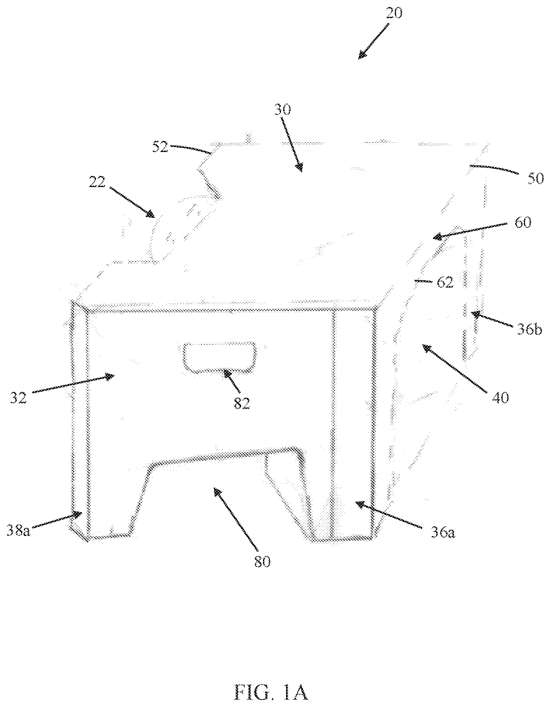

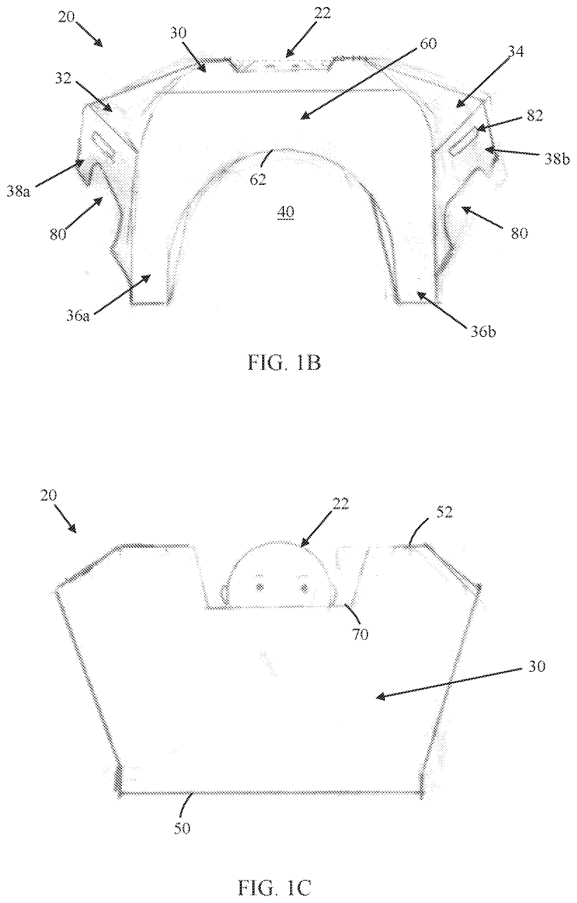

One embodiment of an upper torso restraining device 20 (or “turtle shell restraint”) in accordance with principles of the present disclosure is shown in FIGS. 1A-1C. As a point of reference, FIGS. 1A-1C generally illustrate portions of a subject 22 to whom the upper torso restraining device 20 has been applied. The upper torso restraining device 20 can be, or can be akin to, a shell or shell-like structure, and forms or defines a breastplate 30, opposing first and second side walls 32, 34, first and second bottom pillars 36a, 36b, and first and second top pillars 38a, 38b. Details on the various components are provided below. In general terms, the upper torso restraining device 20 defines an interior region 40 (referenced generally) sized and shaped to receive an upper torso (in a region of the chest and shoulders) or other anatomy of the human adult subject 22. The pillars 36a, 36b, 38a, 38b support the upper torso restraining device 20 relative to a supporting surface such as a floor, ground, gurney, bed, etc. (e.g., when the subject 22 is prone on a floor, the pillars 36a, 36b, 38a, 38b are placed against, thus supporting the restraining device relative to, the floor). The side walls 32, 34, in combination with the corresponding pillars 36a, 38a and 36b, 38b, are sized and shaped for placement over the subject's 22 shoulders. The upper torso restraining device 20 is sized and shaped to pin the subject 22 on the ground surface, and has a sufficiently robust construction such that a user can safely place her or his weight on the breastplate 30 to prevent the subject 22 from moving without endangering the person being restrained or the person applying the device 20. Thus, during use, the upper torso restraining device 20 safely restrains the subject 22 without risk of restriction on the subject's neck or chest that might otherwise lead to or cause asphyxia while providing a physical barrier between user and restrained person.

The breastplate 30 can be a generally flat or planar body, sized and shaped in accordance with typical anatomical form factors for a human adult upper torso. For example, the breastplate 30 can be viewed has having or defining a bottom end 50 opposite a top end 52. The bottom pillars 36a, 36b extend downwardly (relative to the orientation of FIG. 1B) from the bottom end 50. As best shown in FIG. 1B, a spacing between the bottom pillars 36a, 36b, and thus a width of the breastplate 30 along the bottom end 50, is sufficiently sized to be placed over a human adult's chest. In some embodiments, a partial bottom wall 60 extends downwardly (relative to the orientation of FIG. 1B) from the bottom end 50 of the breastplate 30 between the bottom pillars 36a, 36b, and terminates at an edge 62. A spacing between the edge 62 and a lower face of the bottom pillars 36a, 36b is sufficiently sized to receive, or be place over, the subject's upper torso or chest. Moreover, the edge 62 can have the curved or concave (relative to the bottom end 50) shape to better accommodate the normal shape of a human torso.

The breastplate 30 can increase in width from the bottom end 50 to the top end 52 (e.g., a width of the breastplate 30 along the top end 52 can be greater than the width along the bottom end 50), generally mimicking the natural shape of the human anatomy from the torso to the shoulders. As best shown in FIG. 1C, a cut-out 70 can be defined along the top end 52, providing a space or opening for at least a portion of the subject's head when the upper torso restraining device 20 is in place.

A length of the breastplate 30 between the bottom and top ends 50, 52 is selected in accordance with the typical length of the human adult torso from about the base of the rib cage to above the shoulders. The top pillars 38a, 38b extend downwardly from the top end 50. The first side wall 32 projects from the breastplate 30, and extends between and interconnects the first bottom pillar 36a and the first top pillar 38a. Similarly, the second side wall 34 projects from the breastplate 30, and extends between and interconnects the second bottom pillar 36b and the second top pillar 38b. The side walls 32, 34 can have a similar or identical configuration, each forming a recess 80 sized and shaped for placement over a shoulder/upper arm region of the subject 22. In some embodiments, one or both of the side walls 32, 34 can optionally form or carry a handling device 82 (e.g., a cut out, a handle, etc.) conducive to simple grasping by a user's hand.

A length of the pillars 36a, 36b, 38a, 38b in extension from the breastplate 30, as well as dimensions of other openings or open regions of the upper torso restraining device 20 are selected to be tall enough to reach the floor (or other ground surface such as a gurney or bed) while providing sufficient space or clearance for the torso, chest and neck of the subject to be restrained. Various dimensions of the upper torso restraining device 20 can be selected to accommodate an expected size of the subject to be restrained. For example, the upper torso restraining device 20 can be sized and shaped in accordance with anatomical form factors of an adult male. Other dimensions to accommodate other human form factors can be employed. Regardless, the upper torso restraining device 20 is formed (e.g., molded) of a strong, lightweight, structurally robust material (e.g., hardened foam, plastic, etc.). with the resulting structure cable of supporting the weight of a user (e.g., on the order of at least 250 pounds) without collapsing or otherwise failing. In some optional embodiments, at least the breastplate 30 can include or incorporate a bullet-resistant material, such that the upper torso restraining device 20 can be used as a shield in an active shooter scenario. In yet other embodiments, at least the breastplate 30 is clear or transparent or substantially transparent (e.g., within 20 % of truly transparent), affording a user the ability to view the restrained person through the breastplate 30.

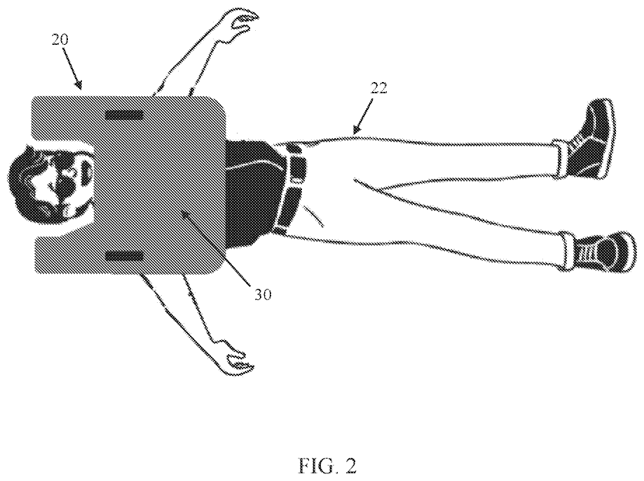

During use, a user (e.g., law enforcement or medical personnel), while holding the upper torso restraining device 20, can approach a standing subject and effect a rapid containment maneuver, similar to capturing a moth in a glass. Regardless, the user can rapidly place the upper torso restraining device 20 over the subject's chest and shoulders to pin them quickly, easily, and safely to a flat surface (e.g., the ground), with the restraining device 20 acting as a shield to the user during the takedown process. Once the subject 22 is on the ground, for example as generally shown in FIG. 2, the user can kneel, sit or stand on the breastplate 30 to prevent the subject 22 from moving. The upper torso restraining device 20 has hollowed out areas for the chest and neck along with areas that contact the shoulders and/or upper arms and extend down to the mid-upper arms to effect a “pin” like hold on the subject (e.g., pressing the subject's shoulder blades to the ground (or other flat surface) and upper arms if possible). The upper torso restraining device 20 can be used to very quickly immobilize a violent/agitated person during a take-down without endangering their breathing or the neck structure (the hollow areas are high enough to not interfere with chest movement while intentionally shielding the chest and neck structurally from contact with the user(s)), and with minimal contact between the user and the subject 22. The upper torso restraining device 20 also provides a hands-free restraint mechanism whereby a user can kneel, sit or even stand on the breastplate 30 while help is on the way and/or a sedative is delivered. Thus, the upper torso restraining device 20 is highly useful in a number of situations. For example, during the often dangerous and violent take-down phase of subduing a patient, suspect, prisoner, active shooter, and general assailants in healthcare settings, schools, malls, police arrests, public spaces, etc. Regardless, the upper torso restraining device 20, as placed over the prone or supine subject, maintains a safe amount of space between the subject (e.g., chest, neck, and head), the upper torso restraining device 20 and the user performing the takedown/restraining, The restraining devices of the present disclosure, such as the restraining device 20, are useful for containing a subject, not necessarily for subduing the subject. The restraining devices of the present disclosure can be used as a “manual hold assist” once the subject has been briefly restrained to the point where the restraining device can be applied. In some examples, multiple devices 20 in the hands of team members can be used/placed over patient hips, arm, one or both legs. Placement of the torso device over both legs at once is another important use for some situations, as on ED gurneys for example, due to their height and narrow shape, teams may choose to use torso devices only over the two legs together and hips (e.g., below the abdomen where pressure could restrict breathing]. In this situation, the use of the torso device 20 over the upper chest may not be feasible, since the patient may be punching with their arms making it hard to achieve the leverage of body weight on a raised surface without injury to staff. This is in contrast to a floor or wall site, where 1-6 devices could potentially be used, one each to contain the torso, hips, each arm, and one or both legs against the flat surface to immobilize.

The upper torso restraining devices of the present disclosure can include or incorporate one or more additional, optional features. For example, the various edges depicted in the view can be rounded. The cut-out 70 along the top end 52 can be minimized or omitted and/or the upper torso restraining device 20 can be positioned relative to the subject 22 such that the top end 52 covers the subject's mouth, serving as a spit guard.

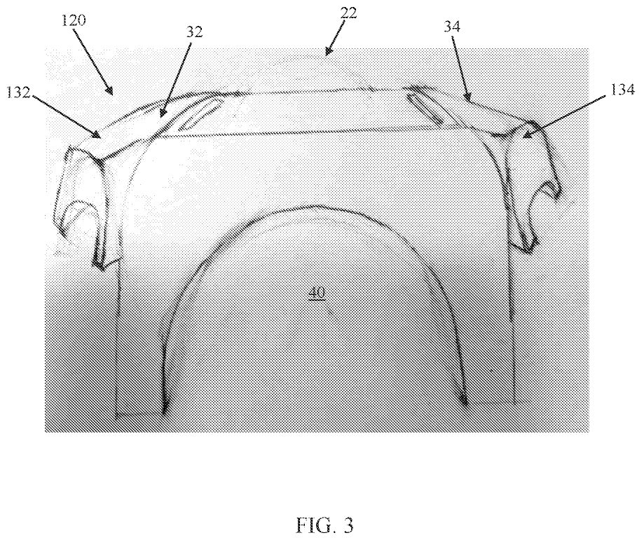

While a shape of the first and second side walls 32, 34 have been shown and described as being sized and shaped to serve as upper arm restraints, other configurations are also acceptable. For example, in other embodiments, the upper torso restraining device can have a more vest-like shape, eliminating one or both of the upper arm restraint configurations of the side walls 32, 34. With this optional design, the subject's upper arm(s) are more readily available for receiving a sedation injection. In yet other embodiments, one or more structures can be formed or provided that are sized and shaped to more extensively capture or cover the upper arm(s). By way of example, FIG. 3 illustrates another embodiment upper torso restraining device 120 in accordance with principles of the present disclosure. The retraining device 120 can be highly akin to the upper torso restraining device 20 (FIG. 1B) described above, having a shell-like shape defining the interior region 40 (referenced generally) sized and shaped to receive an upper torso (in a region of the chest and shoulders) of the human adult subject 22. In addition, the upper torso restraining device 120 includes or defines first and second auxiliary shells 132, 134, extending from, or formed by, the side walls 32, 34, respectively. The auxiliary shells 132, 134 each form a channel that is sized and shaped to receive a corresponding upper arm region of the subject 22. In other embodiments, only one of the auxiliary shells 132, 134 is provided; with these and related embodiments. one upper arm of the subject 22 is restrained, while the other upper arm is exposed or free, for example to receive a sedation injection.

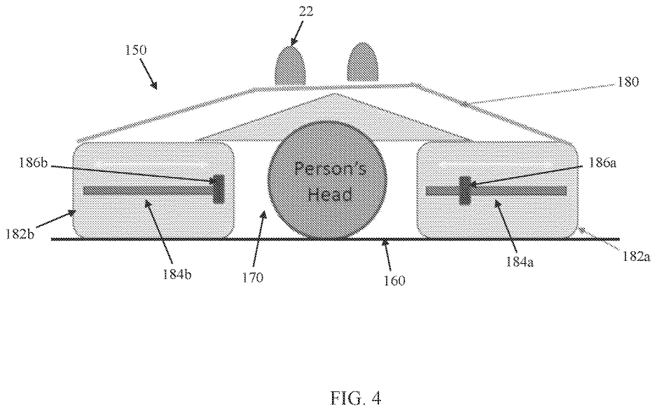

As described above, in some embodiments, the upper torso retraining devices of the present disclosure can be secured over the subject via weight of the user (otherwise sitting, kneeling or even standing on the breastplate). In other embodiments, the upper torso restraining device can include or incorporate features that facilitate a more robust engagement with the floor or other ground surface. For example, another embodiment upper torso restraining device 150 is shown, in simplified form, in FIG. 4. In the view of FIG. 4, the upper torso restraining device 150 has been applied to the subject 22 who is otherwise lying on a floor 160. With this in mind, the upper torso restraining device 150 can generally have any of the shell-like forms described above, defining an interior region 170 sized and shaped for placement over an upper torso of the subject 22, and including a breastplate 180. First and second top pillars 182a, 182b project downwardly from the breastplate 180 in accordance with the descriptions above (it being understood that bottom pillars can also be included, but are hidden in the view of FIG. 4). Each of the top pillars 182a, 182b includes or carries a vacuum mechanism 184a, 184b (referenced generally), configured to create or generate vacuum suction between the top pillar 182a, 182b and the floor 160 when manually actuated by a user as is known in the art. As a point of reference, often floors in hospitals, schools, etc., are very smooth and hard. The vacuum mechanism 184a, 184b is manually operable (via, for example, a slider 186a, 186b) to create vacuum suction between the corresponding top pillar 182a, 182b and the floor 160, thereby better holding the restrained subject 22 to the floor 160. By locating the sliders 186a, 186b above the subject's arms (when the upper torso restraining device 150 has been installed over the subject 22), the subject 22 cannot easily access the sliders 186a, 186b in an attempt to disengage the applied vacuum. A user, however, can quickly access and operate the sliders 186a, 186b as desired, for example to release the applied vacuum to remove the upper torso restraining device 150 from the subject 22.

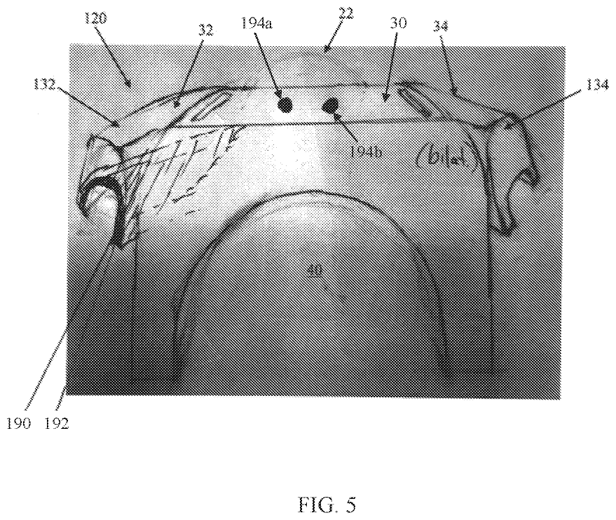

The upper torso restraining devices of the present disclosure can optionally include one or more additional features that can further enhance immobilization and/or reduce a risk of injury to a person being constrained. For example, another embodiment upper torso restraining device 190 is shown in FIG. 5 applied to the subject 22. The upper torso restraining device 190 can include any of the features described above (e.g., can be generally akin to the upper torso restraining device of FIG. 3). In addition, the upper torso restraining device 190 can include one or both of an inflatable bladder 192 and/or a cushion layer 194. Where provided, the inflatable bladder 192 is secured to an interior surface of the device, for example at or along one or more of the breastplate 30, the side walls 32, 34, the auxiliary shells 132, 134, etc. In some embodiments, two or more inflatable bladders can be provided. Regardless, the inflatable bladder(s) 192 can be fluidly connected to a rapid inflation/deflation system, having user operated buttons 196a, 196b, the user-actuation of which causes the inflatable bladder 192 to inflate or deflate. The cushion layer 194 can be formed of a soft material (e.g., soft foam) and is secured over a surface inflatable bladder 192, providing a soft contact surface for the subject 22 when constrained. In other embodiments in which the inflatable bladder 192 is omitted, the cushion layer 194 can be applied directly to an interior surface of the device.

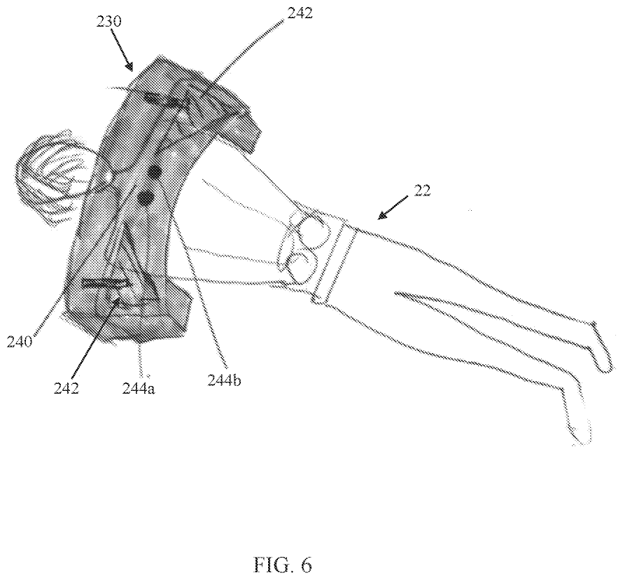

While the upper torso restraining devices have been shown and described for placement over a supine human subject (i.e., lying on the back), other configurations and end-use applications are also envisioned. For example, FIG. 6 illustrates another upper torso restraining device 230 in accordance with principles of the present disclosure and useful, for example, in constraining the subject 22 when prone (i.e., lying flat with the face downward). In general terms, the upper torso retraining device 230 can have any of the constructions described above, include or provide a top plate or wall 240 (e.g., akin to the breastplate 30 of the upper torso restraining device 20 (FIG. 1)) supported by two or more pillars that combine to define a shell or cavity sized and shaped for placement over the upper back, neck, and shoulders of the prone subject 22. A size of the shell can accommodate, in some examples, the subject 22 while handcuffed. Regardless, the upper torso restraining device 230 has sufficient structural strength to support, and not buckle or collapse under the weight of, a user kneeling, sitting or standing on the top plate 240.

The upper torso restraining device 230 can optionally include one or more additional features that further enhance immobilization and/or reduce a risk of injury to the constrained subject 22. For example, the upper torso restraining device 230 can include inflatable bladders 242. Where provided, the inflatable bladders 242 are secured to an interior surface of the shell, for example at or along opposing side regions that otherwise correspond to a location of the subject's shoulders when the device 230 is applied. Regardless, the inflatable bladders 242 can be fluidly connected to a rapid inflation/deflation system (e.g., including one or two air pumps hidden inside a structure of the device 230 similar to a small, air mattress pump), having user operated buttons 244a, 244b, the user-actuation of which causes the inflatable bladders 242 to inflate or deflate. When inflated, the bladders 242 can achieve a more snug fit to the subject 22, and can be useful, for example, with smaller individuals. The inflatable bladders 242 can be covered with padding, a cushion or a cushion-like material (e.g., akin to a blood pressure cuff material). Optionally, other interior surfaces of the shell can be covered by a cushion or padding material (e.g., soft foam, egg crate material, etc.), providing a soft contact surface for the subject 22 when constrained.

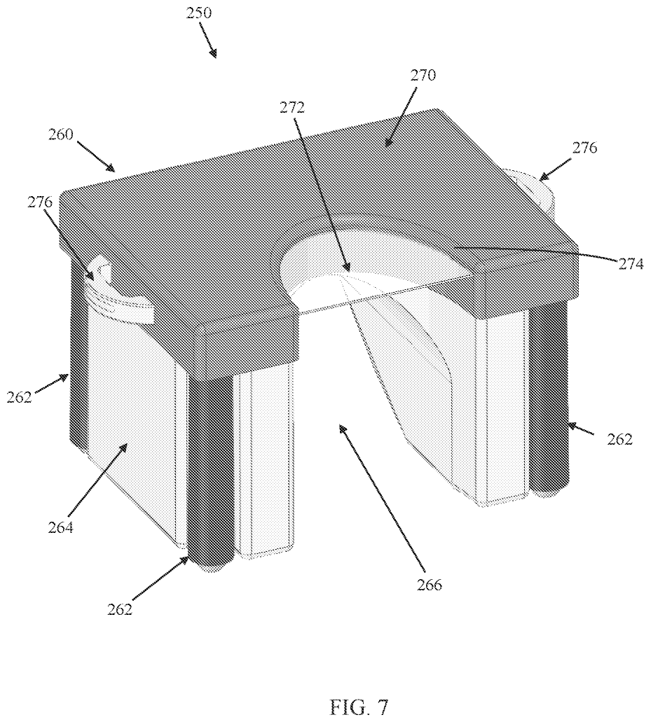

Another embodiment of a restraining device 250 in accordance with principles of the present disclosure and useful, for example, as an upper torso restraining device is shown in FIG. 7. The restraining device 250 includes a platform assembly 260, pillars 262, and a core 264. Details on the various components are provided below. In general terms, the pillars 262 extend from the platform assembly 260 to collectively define an open space within which the core 264 is maintained. The core 264, in turn, defines an interior region 266 (referenced generally) sized and shaped to receive a head, neck, and upper torso (chest and shoulders) of the human adult subject. During use, the pillars 262 support the restraining device 250 relative to a flat or supporting surface (e.g., ground, bed, gurney, backboard, etc.). For example, when the subject is prone on a floor or other supporting surface, the pillars 262 are placed against or onto the floor. The restraining device 250 is sized and shaped to pin the subject on the supporting surface, and has a sufficiently robust construction such that a user can safely place her or his weight on the platform assembly 260 to prevent the subject from moving without endangering the subject being restrained or the user applying the device 250.

The platform assembly 260 can assume various forms, and in some embodiments includes a platform or breastplate 270 and a window 272. The platform 270 is generally constructed to receive a weight of a user (e.g., a user can kneel on the platform 270 when restraining subject), and can include or carry padding or similar material for comfortable, prolonged interface by a user. For example, the platform 270 can optionally be made of a lightweight, structurally robust material such as metal, carbon fiber, titanium, etc., to which a cushioning material or body is applied. A cutout 274 is formed at a leading side 276 of the platform 270. A size and shape of the cutout 274 corresponds with a size and shape of the window 272. The window 272 is assembled to the platform 270 at the cutout 274 and can be formed of an optically transparent or substantially transparent material (e.g., clear or semi-clear plexiglass or other plastic). In some embodiments, the window 272 is configured to provide added protection to a user, for example impact resistance (e.g., bullet proof) such as by utilizing a bullet resistant polycarbonate or acrylic sheeting. The platform assembly 260 can optionally include one or more additional components or features, such as handles 276. While FIG. 7 generally reflects the platform assembly 260 and the pillars 262 as being sized and shaped to arrange the platform 270 parallel to the flat surface on which the pillars 262 are placed, other configurations are also acceptable. For example, the retraining device 250 can be configured to angle the platform 270 to some degree (e.g., 10-15 degrees) relative to the flat surface on which the pillars 262 are placed; these and related embodiments can be beneficial for users and situations to allow greater stability for the user kneeling on the platform 270.

The core 264 can be formed of a conformable material (e.g., foam, over-molded cushioning material, etc.) configured to bear against, and conform to, an anatomy of the subject being constrained as further explained below. In some embodiments, outer dimensions of the core 264 generally correspond to dimensions of the platform 270 and the pillars 262 to provide the restraining device 250 with a compact, overall footprint for ease of handling and use. For example, the core 264 can be viewed as defining a front end opposite a back end, and a first side opposite a second side. Contours of the interior region 266 generally expand in width from the front end to the back end. In some instances, the front end will generally be located near or over a head/neck region of the subject, while the back end will be located near or over an upper torso region of the subject. However, the restraining device 250 (and thus the core 264) is equally well-suited for use in immobilizing other anatomy of a subject (e.g., hips, arm(s), leg(s)) alone or in combination with the torso. In addition to the interior region 266, other recesses can be formed in the core 264 to accommodate features of the restraining device 250. A slot can be formed at each corner of the core 264 to accommodate a corresponding one of the pillars 262. A recess 292 can be formed through an upper surface of the core 264 that is open to the front end and the interior region 266. The recess is sized and shaped to correspond with the cutout 274, and thus the window 272. Upon final assembly, then, and as reflected by FIG. 7, a user can visualize the interior region 266 (and thus a subject otherwise located within the interior region 266) from above the restraining device 250 via the window 272.

During use, a user (e.g., law enforcement or medical personnel), while holding the restraining device 250 (e.g., at the handles 276), can approach a subject and effect a rapid containment maneuver. Once the subject is on the ground (or other supporting surface), for example in a supine position, the restraining device 250 is placed over a desired anatomical area of the subject (e.g., the upper torso, neck and head), bringing the core 264 into direct contact with the subject. Opposing ones of the pillars 262 can be located above and below a shoulder region of the subject, with the core 264 readily conforming to an anatomy of the subject. The restraining device 250 can be arranged relative to the subject such that the subject's head is readily visible through the window 272. The user can then kneel, sit or stand on the platform 270 to prevent the subject from moving (with the subject being restrained by the core 264 and the user's weight). While the subject's shoulders are effectively pinned against the supporting surface, the interior region 266 is sized and shaped to provide clearance over the chest and neck areas of the subject so that the subject can freely or easily breathe. Depending upon the environment in which the subject is being constrained, the restraining device 250 can be strapped into place over the subject. For example, where the subject 22 is constrained on a gurney, bed, or similar supporting surface, straps can be placed through the handles 276 (FIG. 7) to secure the restraining device 250 to the supporting surface.

The restraining device 250 is equally useful when applied to other anatomical regions of the subject and/or with the subject in a different position. For example, the retraining device 250 can be used to control a subject in a prone position.

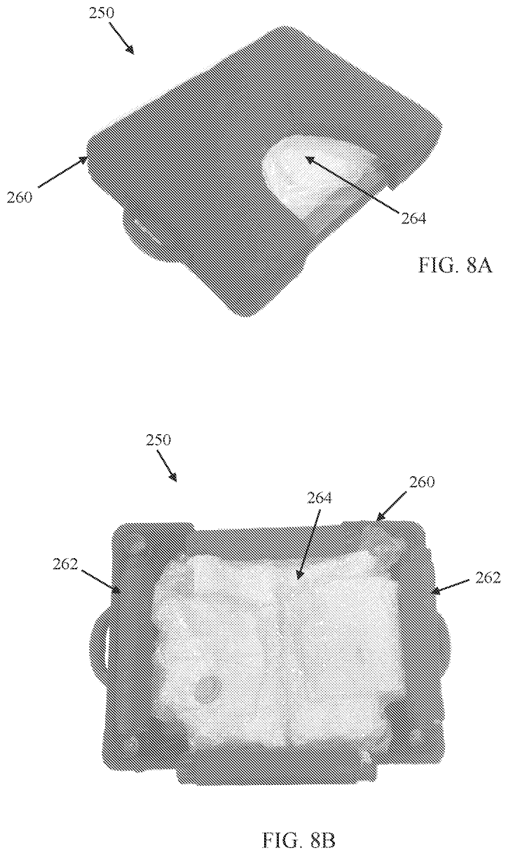

In some embodiments, the restraining device 250 is configured to be transitioned to the deployed or use state shown from a storage or collapsed state. For example, and with additional reference to FIGS. 8A and 8B, the pillars 262 can be releasably or pivotably attached to the platform assembly 260. Similarly, the core 264 can be removable relative to the platform assembly 260 and the pillars 262. In this regard, with optional embodiments in which the core 264 is formed of a foam or similar material, the core 264 can be held in collapsed or constrained arrangement in the storage state. For example, the core 264 can be held or stored in a collapsed or flattened state within a container 320 (e.g., a vacuum-seal bag). In some examples, a size of the core 264 in the collapsed state is at least three times less than the size in the normal or unconstrained state. In some non-limiting examples, the core 264 can have a height on the order of 16 inches in the normal or unconstrained state, and a height on the order of 3 inches in the collapsed state. Regardless, with these and related embodiments, the storage state of the retraining device 250 can include the disassembled (or folded) pillars 262 and the collapsed/flattened core 264 secured within an interior of the platform assembly 260 as shown in FIGS. 8A and 8B.

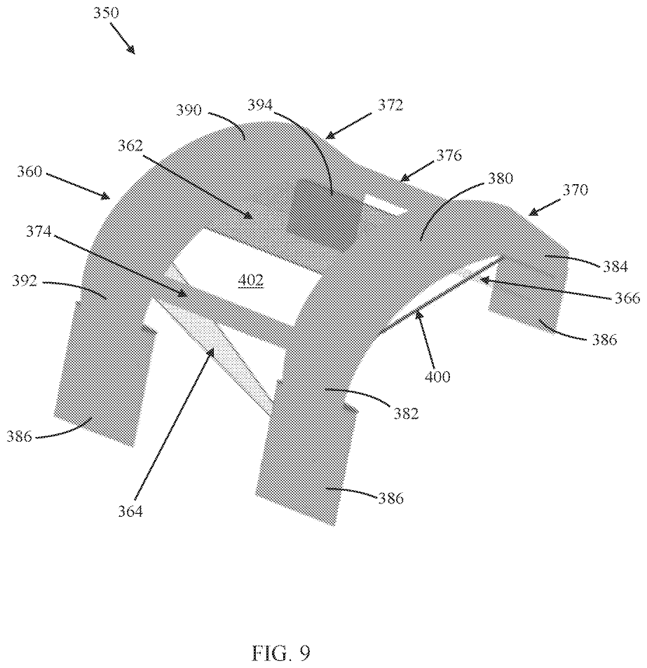

Another embodiment of a restraining device 350 in accordance with principles of the present disclosure and useful, for example, as an upper torso restraining device is shown in FIG. 9. The restraining device 350 includes framework 360 (referenced generally), a kneeling platform or breastplate 362, and first and second shoulder straps 364, 366. As a point of reference, the platform 362 is illustrated as being partially transparent in the view of FIG. 9 for ease of understanding.

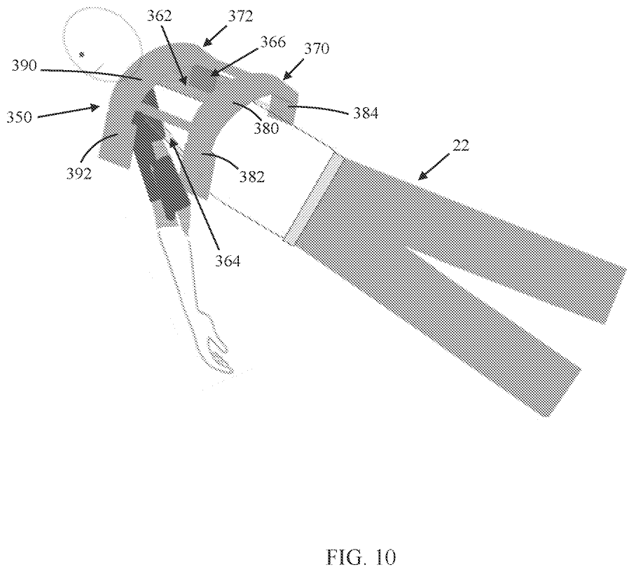

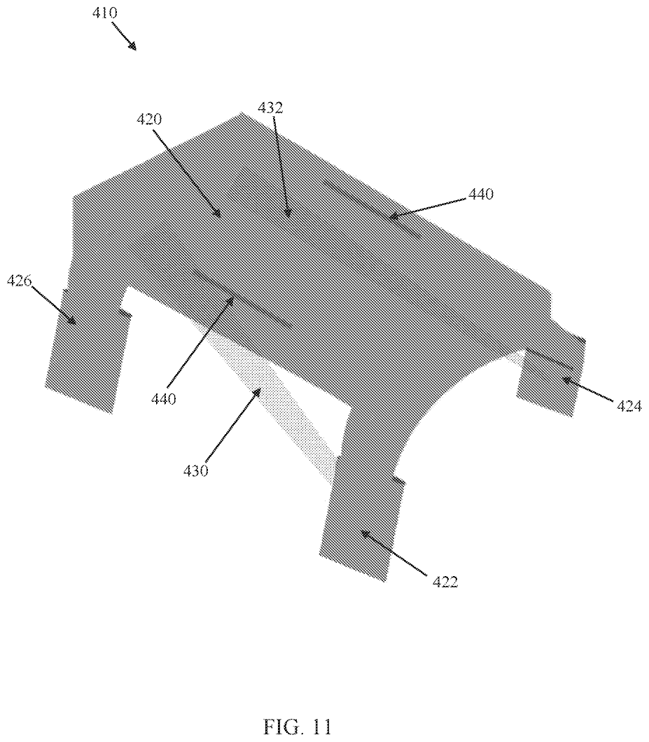

The framework 360 can be configured to readily transition between a storage state (not shown) and the deployed state of FIG. 9. The framework 360 forms or defines a trailing frame member 370, a leading frame member 372, and first and second cross members 374, 376. The cross members 374, 376 extend between and interconnect the trailing and leading frame members 370, 372, maintaining the frame members 370, 372 in the spaced apart arrangement shown. In at least the deployed state, the trailing frame member 370 forms or defines a central region 380 and opposing, first and second pillars 382, 384. The central region 380 can have the arch-like shape illustrated in FIG. 10. The first and second pillars 382, 384 extend from opposite sides of the central region 380, and terminate at an end section 386 that is generally configured for secure placement against a surface during use. In this regard, a shape and size of the end section 386 provides sufficient surface area for supporting the restraining device 350 under expected forces (e.g., weight of an adult). In some embodiments, however, a width of each of the pillars 382, 384 is sufficiently narrow to fit between the upper arms and trunk of an adult to facilitate handcuffing if necessary. While the central region 380 and the pillars 382, 384 have been shown and described as being defined by a single, continuous body, in other embodiments, one or both of the pillars 382, 384 can be formed apart from, and subsequently assembled to, the central region 380. The leading frame member 372 can be substantially identical to the trailing frame member 370, forming or defining a central region 390 (that is optionally arch-shaped), and opposing pillars 392, 394.

The cross members 374, 376 can have substantially identical constructions, and generally rigidly extend between the central regions 380, 390 at opposite extents thereof. For example, the first cross member 374 can extend from a general point of demarcation of the first pillar 382 from the central region 380 of the trailing frame member 370 to a corresponding location along the leading fame member 372. As described in greater detail below, a shape and arrangement of the cross members 374, 376 relative to the frame members 370, 372 provide convenient grasping surfaces for a user of the restraining device 350. Thus, in some embodiments, the cross members 374, 376 can serve has handles or grips for a user.

In some embodiments, the framework 360 is configured to be lightweight yet structurally robust, for example formed of aluminum or similar metal or metal-like material such as carbon fiber, titanium, etc., to which a cushioning material or body is optionally applied. A cross-section shape of the frame members 370, 372 and the cross member 374, 376 is selected to maintain a shape of the deployed state under expected forces as described below. In this regard, an overall strength of the framework 360 can be enhanced via the optional arch-like or curved shape of the central regions 380, 390.

As mentioned above, in some embodiments the framework 360 is configured to be transitioned to (or from) the deployed state of FIG. 9 to a storage state. In the storage state, at least two, optionally all, of the pillars 382, 384, 392, 394 are pivoted inwardly, toward the corresponding central region 380, 390. In some embodiments, the framework 360 is constructed such that at least two, optionally all, of the pillars 382, 384, 392, 394 are effectively spring-loaded (e.g., via a shape of the framework 360, an auxiliary mechanism, etc.), and can be biased to the self-assume the deployed state. For example, just the two pillars 392, 394 are attached to the straps 364, 366, respectively. During use, all four pillars 382, 384, 392, 394 can be placed against a surface while the front two pillars 392, 394 near the subject's shoulders can be lowered mechanically to bring the straps 364, 366 securely in contact with the ground. In yet other embodiments, the framework 360 can be constructed such that one or more or all of the pillars 382, 384, 392, 394 are extendable/retractable relative to the corresponding central region 380, 390 Regardless, with embodiments in which the framework 360 permits transitioning between the deployed state and a storage state, one or more mechanisms can be provided that hold or lock the pillars 382, 384, 392, 394 relative to the corresponding central region 380, 390 in one or both of the deployed and storage states. For example, a mechanism(s) (not shown) can be provided that retain the pillars 382, 384, 392, 394 in the storage state (e.g., a spring-loaded, ratcheting-type mechanism). An actuator (e.g., button or similar device) can be carried by one or both of the cross members 374, 376. When actuated by a user, the mechanism(s) is prompted to release or extend the pillars 382, 384, 392, 394 to the deployed state, with the pillars 382, 384, 392, 394 rigidly or robustly maintaining or locking in the deployed state. Other configurations can alternatively be employed.

Regardless of whether or not the framework 360 is transitionable from the deployed state to a storage state, in the deployed state of FIG. 9, a distance between the pillar ends 386 and the corresponding central region 380, 390 is sufficiently sized for placement over a subject (e.g., generally defining or creating interior region sized and shaped for placement over an upper torso of the subject). In some embodiments, the retraining device 350 can optionally be configured such that a transverse distance between the pillars 382, 384 and the pillars 392, 394 is adjustable. For example, a sliding mechanism 400 can be provided with the trailing frame member 370 that is configured to shorten or widen the transverse distance between the pillars 382, 384 in the deployed state. With these and related embodiments, an actuator (not shown) can be provided (e.g., along one or both of the cross members 374, 376), user prompted operation of which locks the sliding mechanism 400 upon attaining a desired distance between the pillars 382, 384. Though not shown, a similar sliding mechanism (and corresponding actuator) can be provided with the leading frame member 372 for adjusting a distance between the pillars 392, 394 in the deployed state. With embodiments in which the distance between the pillars 382, 384 and the pillars 392, 394 can be adjusted, the restraining device 350 can quickly be arranged to a size appropriate for a particular subject and moreover, a desired anatomy of the subject to be constrained. That is to say, the restraining device 350 can more universally be applied to a chest, back, waist. arms, legs, etc., of a subject.

The platform 362 can take various forms, and is mounted to the central regions 380, 390 as shown (e.g., at a top of the framework 360). The platform 362 is generally constructed to receive a weight of a user (e.g., a user can kneel on the platform 362 when restraining subject), and can include or carry padding or similar material for comfortable, prolonged interface by a user and for protection for users and the individuals being contained should the device be used by an individual to attempt to contact and/or injure one or more users. In some embodiments, a footprint of the platform 362 is less than an available area between the central regions 380, 390 and between the cross members 374, 376. With these non-limiting examples, one or more open regions or spaces (one of which is labeled at 402 in FIG. 9) is provided along the restraining device 350 and through which a subject being restrained can be visualized.

The shoulder straps 364, 366 can assume various forms, and are generally configured for contacting and restraining, a subject with minimal pain. For example, the shoulder straps 364, 366 can each be akin to a backpack strap, including a base nylon or polyester webbing or similar durable, high tensile strength material (e.g., leather) covered with a padding (e.g., foam, cloth, etc.). In other example, the straps 364, 366 can be a lightweight material such as fabric or flexible thin plastic in “billows” shape connect straps to the platform above to prevent the person being restrained from getting a strong grip around the strap during the containment event. Regardless, the straps 364, 366 are connected to the framework 360 so as to extend below the platform 362. In some examples, the first shoulder strap 364 extends from the first pillar 382 of the trailing frame member 370 to the central region 390 of the leading frame member 372 at one side of the platform 362, and the second shoulder strap 366 extends from the second pillar 384 of the trailing frame member 370 to the central region 390 of the leading frame member 372 at an opposite side of the platform 362. As made clear below, this one acceptable configuration readily arranges the shoulder straps 364, 366 over the shoulders of a subject during use. In some embodiments, a point of connection between the shoulder straps 364, 366 and the framework 360, for example at the leading frame members 372, is adjustable via a mechanism (e.g., a slidable, ratchet-type or latch-type mechanism), allowing a user to raise or lower the shoulder straps 364, 366 relative to the platform 362 to better accommodate a size of a particular subject. With these and related embodiments, the straps 364, 366 can, for example, slide along the leading frame members 372 from a released state to a locked state.

During use, a user (e.g., law enforcement or medical personnel), while holding the restraining device 350 (e.g., at the cross member 374, 376), can approach a subject, quickly actuate the restraining device 350 to the deployed state, and effect a rapid containment maneuver, similar to capturing a moth in a glass. Once the subject 22 is on the ground, for example as generally shown in FIG. 10, the restraining device 350 is placed over a desired anatomical area of the subject 22 (e.g., the upper torso in the non-limiting example of FIG. 10), bringing the shoulder straps 364, 366 into direct contact with the subject 22. With optional embodiments in which the restraining device 350 provides one or more size adjustment features (e.g., adjustable distance between the pillars 382, 384), the user can arrange the restraining device 350 to a desired size for the subject/anatomical location. The user can then kneel, sit or stand on the platform 362 to prevent the subject 22 from moving (with the subject being restrained by the shoulder straps 364, 366). With embodiments in which the shoulder straps 364, 366 extend from the pillars 382, 384, respectively, of the trailing frame member 370 to the central region 390 of the leading frame member 372, a weight of the user on the platform 362 effectively places the shoulder straps 364, 366 into tension; this tension serves to move or push the shoulder straps 364, 366 upwardly along the central region 390 into a locked position relative to the subject 22. With the arrangement of FIG. 10, only a relatively small force or pressure need be applied to the shoulders (or other anatomy) of the subject 22 by the shoulder straps 364, 366; a retraining force sufficient to prevent the subject 22 from leveraging against the weight of the user. The curved shape of the central regions 380, 390 contributes to an overall strength of the restraining device 350 against the weight of the user while providing sufficient space for the neck and breathing of the subject 22. Spacing between the first pillars 382, 392 (and the second pillars (hidden in FIG. 10)) is appropriate for an arm of the subject 22 to pass through. Ends of the straps 364, 366 can be raised and lowered tailorable to an individual's size to provide snug contact against shoulders and/or upper arms. Importantly, the straps 364, 366 are prevented within the framework 360 from being lowered below the neck width of an average adult so that were the device be in the hands of an agitated individual it could not be used to suffocate others through rotation of the device and placement of the strap over the neck region. This fixed minimum height from straps 364, 366 to the flat surface, approximately 6 inches in some examples, would allow a user or other person being assaulted with the device rotated 90 degrees to turn their head to the side underneath the strap 364, 366 with structurally protected space for breathing, in case of attempted misuse of the device by an agitated individual. Importantly, the height of the foam insert within the framework 360 and under the breastplate for holding pressure on the individual's shoulders and/or upper arms against the flat surface would also provide this protective function at suffocation, i.e., building with a soft foam layer on the bottom surface of the foam insert that would not compress the neck to the point of difficulty breathing if the device were used improperly.

Another embodiment of a restraining device 410 in accordance with principles of the present disclosure and useful, for example, as an upper torso restraining device is shown in FIG. 11. The restraining device 410 is akin to the restraining device 350 (FIG. 9) described above and includes a kneeling platform or breastplate 420, first and second trailing pillars 422, 424, first and second leading pillars 426 (it being understood that the second leading pillar is hidden in the view) and first and second shoulder straps 430, 432. As a point of reference, the platform 420 is illustrated as being partially transparent in the view of FIG. 11 for ease of understanding.

In some embodiments, the restraining device 410 can be a flat-top version of the restraining device 350 (FIG. 9). The pillars 422-426 are formed as extensions of the platform 420, and in some embodiments are configured to be transitionable from a storage state to the deployed state of FIG. 11. In some examples, the restraining device 410 can include appropriate mechanism(s) and corresponding actuator(s) that allow a user to quickly deploy the pillars 422-426 to the deployed state. In other embodiments, an arrangement of the shoulder straps 430, 432 can assist or effect deployment of one or more or all of the pillars 422-426. For example, the shoulder straps 430, 432 (e.g., padded nylon webbing) can extend from a respective one of the trailing pillars 422, 424 to an underside of the platform 420 as shown. Once the shoulder straps 430, 432 are placed in contact with a subject and a user's weight is placed onto the platform 420, the shoulder straps 430, 432 force the trailing pillars 422, 424 to the deployed state as shown. Regardless, the shoulder straps 430, 432 are pushed upward into a locked position through the weight of the user on the platform 420.

One or more additional features can optionally be provided with the restraining device 410. For example, handles 440 can be provided with the platform 420 for case of handling. One or more mechanisms can be provided for adjusting a transverse distance between the trailing pillars 422, 424 and the leading pillars 426 (e.g., akin to the sliding mechanism 400 (FIG. 9) described above).

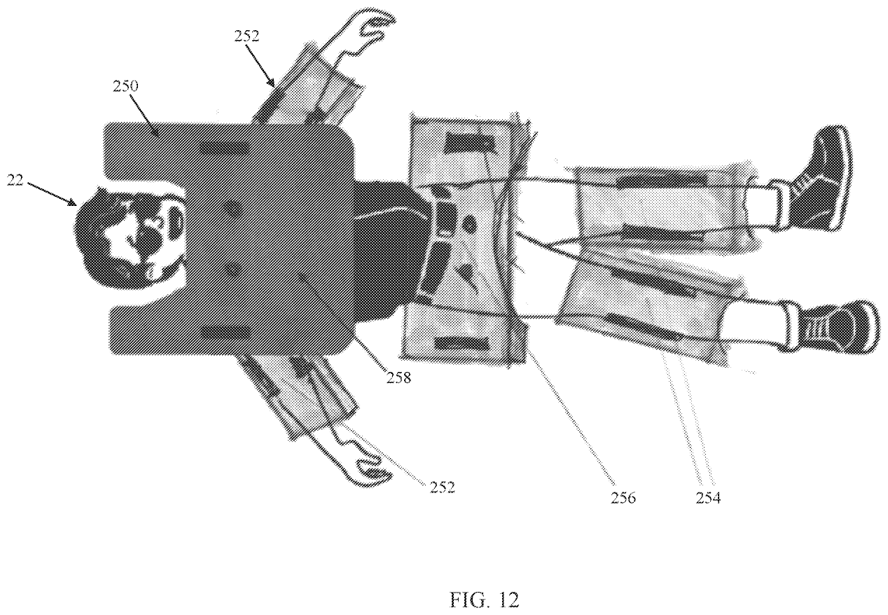

The upper torso restraining devices of the present disclosure can, in some embodiments, be used alone to restrain a subject. In other embodiments, systems of the present disclosure can further include one or more limb restraining devices to cover and/or surround the joints (knees and elbows) that a thrashing subject might use to punch and/or kick at healthcare/security personnel. This provides safety both for the subject, who is usually being laid upon in the chest, legs, and arms when trying to subdue them. In some examples, and with reference to FIG. 12, systems of the present disclosure can include, in addition to an upper torso retraining device 250, one or more limb restraining devices 252 appropriate for a subject's arm(s) or elbow joint(s), one or limb restraining devices 254 appropriate for a subject's leg(s) or knee(s), and a lower torso/hips at or below the belt line restraining device 256. In general terms, the restraining devices 250-256 can be highly akin to the upper torso restraining device 20 as described above, but sized/shaped for a designated anatomy. As with the upper torso restraining device 20, the restraining devices 200-204 include or provide a rigid wall 258 (e.g., akin to the breastplate 30 of the upper torso restraining device 20) supported by two or more pillars that combine to define a shell or cavity sized for placement over the designated anatomy. With this configuration, once applied to the subject 22, a user can kneel, sit or stand on the retraining devices 250-256 to better immobilize the subject 22. One or more of the restraining devices 250-256 can incorporate additional features that facilitate safe and secure application to the intended anatomy. For example, the hip restraining device 256 can have an arch-like shape to limit pressure being applied to the lower abdomen of the subject 22, can be lined with foam or other cushion-type material (e.g., an egg crate material, mattress material, cushion, etc.), can include or carry an inflatable bladder for generating a snug fit to the subject 22, etc. In some embodiments, two or more of the restraints 250-256 can be used together as part of systematic method for containing the subject 22. Multiple personnel, each responsible for one (or perhaps more) of the restraints 250-256, containing/restraining the subject 22 against a firm surface as generally reflected by FIG. 12.

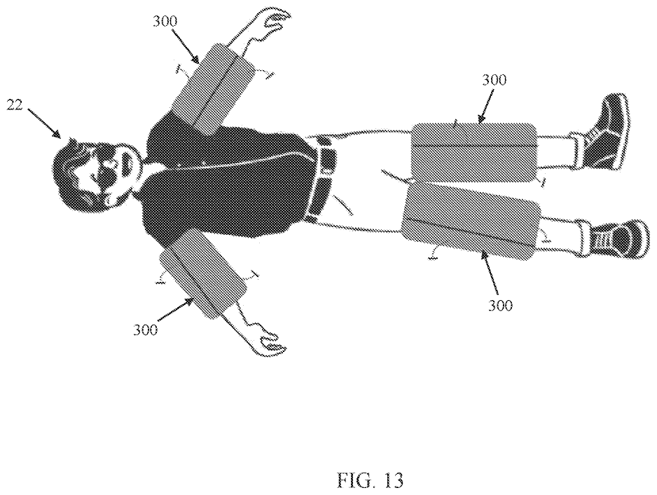

The limb restraining devices of the present disclosure can have other forms. Another embodiment of four limb restraining devices 300 in accordance with principles of the present disclosure is shown in FIG. 13 as applied to the subject 22. In general terms, the limb restraining device 3000 is configured as a rapidly-inflatable restraint. As described below, the limb restraining device 300 can include a dual rapid inflation option (e.g., both CO₂ canisters and a reusable approach by air compressor), rapid inflation ports (for quick hands-on-hands-off code green team inflation and deflation using a small portable air compressor), etc. The limb restraining devices 300 help rapidly immobilize a violent/agitated subject during a takedown by using inflation to straighten and hold limbs that are often flexed, punching, kicking, and with elbows and knees which staff are hesitant to hold in fear of injuring the subject.

For example, the limb restraining device 300 can include an inner layer and an outer layer each formed of a flexible, air impervious material. The inner layer is intended to be placed against the subject during use, with the outer layer thus being exposed. In other embodiments, one or both of the inner and outer layers can be formed of, or covered by, a plastic, canvas or other durable material. In yet other embodiments, one or both of the layers can be clear or transparent or substantially transparent to allow for viewing the subject to which the limb retaining device 300 is applied (e.g., for assessing breathing and circulation). As described in greater detail below, an internal inflation region is defined between the inner and outer layers (e.g., the inner and outer layers can be secured to one another along the perimeters thereof in a sealed manner). Complementary fasteners or fastening materials (e.g., hook-and-loop type complementary fastener materials such as Velcro®) can be provided on the inner and outer layers, respectively. With this configuration, the limb restraining device 300 can be wrapped about a subject's limb, with the complementary fasteners brought into engagement with one another to secure the wrapped limb restraining device 300 in place. In this regard, the limb restraining device 300 can have a relatively large size that facilitates quick wrapping about a bent leg or arm of a subject.

One or more rapid inflation port assemblies are carried by, and accessible at an exterior of, the outer layer. The rapid inflation port assemblies are configured for quick insertion of an inflation device nozzle, for example a portable air compressor nozzle. The limb restraining device 300 can also be configured for rapid self-inflation or auto-inflation via manual actuation (e.g., pulling) of one or more pull cords.

In general terms, the rapid inflation port assembly can, in some examples, include or provides a port tube and a diffuser body. The port tube is fluidly open to an exterior of the outer layer and the diffuser body. The diffuser body is secured to an interior of the inner layer and forms plurality of air holes. A self-sealing membrane is carried within, or formed by, the port tube. With this configuration, an inflation device nozzle (not shown) can be inserted into the port tube and into or through the membrane. Air flow from the nozzle is delivered to the diffuser body and exits into an interior region, causing the limb restraining device 300 to inflate (forcing the inner and outer layers away from one another). The limb restraining device 300 can be deflated in a reverse fashion. In some embodiments, the inflation port assembly can further include a funnel secured to the port tube and exposed outside the outer layer. Where provided, the funnel provides a convenient surface for more quickly guiding the inflation device nozzle into the port tube. In some related embodiments, the funnel can be releasably secured to the port tube (e.g., a threaded-type engagement).

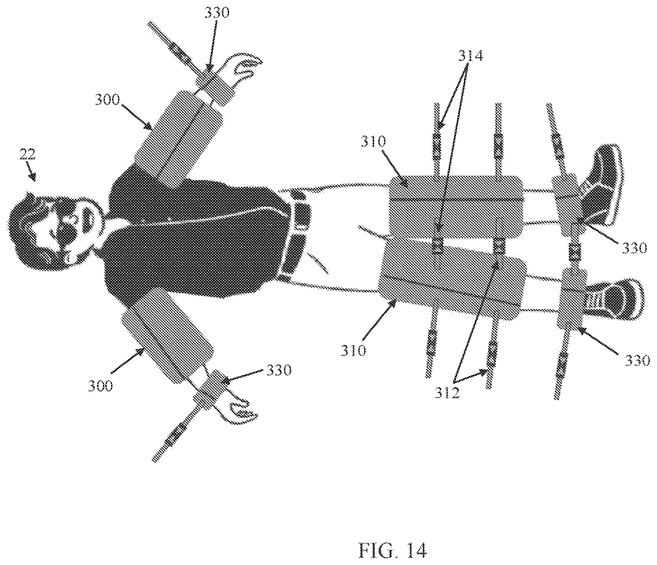

The limb restraining devices of the present disclosure can optionally include one or more additional features. For example, in some embodiments the limb restraining devices of the present disclosure include or incorporate two or more air bladders (e.g., layered air bladders) for durability and redundancy (e.g., an inner inflatable bladder with an outer inflatable bladder in case a leak occurs at the inner bladder). In yet other embodiments, and with reference to FIG. 14, two limb restraining devices 310 are shown applied over the knees/legs of the subject 22. The limb restraining device 310 can be highly akin to the limb restraining device 300 (FIG. 13) described above, and are generally configured to be wrapped about the subject 22 and inflated. In addition, the limb restraining devices 310 include or carry one or more straps 312. The strap(s) 312 are secured to and extend from the outer layer of the corresponding limb restraining device 310 and can include a quick-release buckle 314. With this configuration, the straps 312 from the two limb restraining devices 310 can be coupled to one another (e.g., between the subject's forearms and/or between the subject's shins or thighs). Alternatively or in addition, the strap 312 can be coupled to another object 320 as shown in FIG. 15, such as a firm backboard, ambulance gurney, ER gurney, bed, etc. Regardless, the so-coupled straps 312 can provide additional restraints on movement by the subject. As a point of reference, FIGS. 14 and 15 further illustrate cuff-type limb restraining devices 330 secured about the wrists and ankles of the subject 22. The cuff-type limb restraining devices 330 are highly akin to the limb restraining devices described above, but have a smaller size that more closely approximate a size of the human wrist or ankle. The inflatable cuff-type ankle and wrist restraints 330 with quick release plastic buckles can provide redundancy in the system for the rapidly inflatable large joint immobilizers in case deflation occurred accidentally. There could be other straps in place as well in case this occurred, as placement of these additional straps would be a safe and easy process once the subject 22 was subdued.

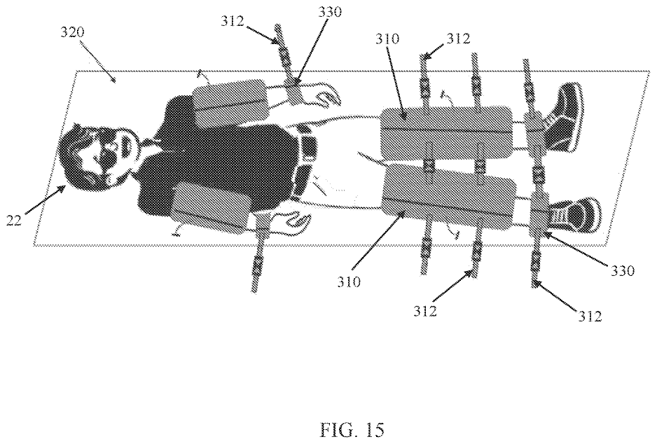

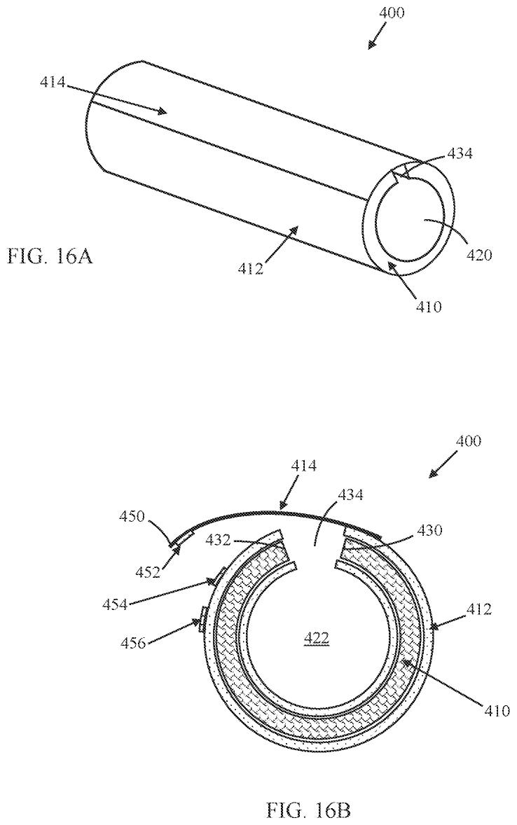

Another example of a limb restraining device 400 in accordance with principles of the present disclosure is shown in FIGS. 16A and 16B. The limb restraining device 400 includes a core 410, a liner 412, and a flap 414. Details on the various components are provided below. In general terms, the core 410 is sized and shaped for placement over and/or around a subject's limb. The liner 412 covers the core 410. The flap 414 extends from an exterior surface of the liner 412; the liner 412 and the flap 414 can provide convenient surfaces or features that promote a releasable, snug fit of the limb restraining device 400 about the subject's limb. The limb restraining device 400 can optionally including padding (e.g., removable padding) to further promote atraumatic yet snug fit to the subject's limb.

The core 410 is formed to have a generally tubular shape, terminating at opposing, open ends 420 (one of which is visible in FIG. 16A). As best shown in FIG. 16B, the core 410 forms an incomplete circle in transverse cross-section to define an interior region 422, with opposing edges 430, 432 of the core 410 being separated by a gap 434 along an entire length of the core 410. The core 410 has a strong, lightweight, and compliant construction that maintains the incomplete circular shape, but can readily be forced to larger and smaller sizes or diameters (i.e., transitioned from the size or diameter reflected by FIG. 16B). More particularly, a construction of the core 410 permits the opposing edges 430, 432 to be forced toward one another with the potential of the edges 430, 432 overlapping to reduce a size of the gap 434, thus reducing a size or diameter of the interior region 422. Conversely, a construction of the core 410 permits the opposing edges 430, 432 to be forced away from one another to increase a size of the gap 434, thus increasing a size or diameter of the interior region 422. With this construction, the opposing edges 430, 432 can be forced away from one another to facilitate easy insertion of a subject's limb into the interior region 422 via the now-enlarged gap 434. Alternatively or in addition, the subject's limb can be slid into the interior region 422 via the open ends 420. Regardless, once the subject's limb is within the interior region 422, the opposing edges 430, 432 can be forced toward one another such that the limb restraining device 400 is rendered snug over the subject's limb.

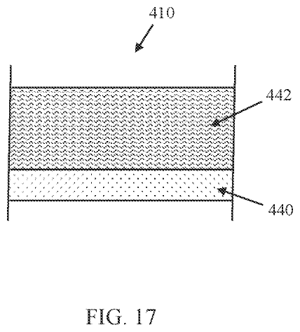

The core 410 can be formed of a variety of material appropriate for constraining a subject's limb in a relatively non-traumatic manner. In some embodiments, the core 410 is or include a foam material (e.g., a polyethylene foam commonly used with pool noodles). With the non-limiting example FIG. 17, the core 410 includes first and second layers 440, 442. The first layer 440 can have stiffer construction (as compared to a stiffness of the second layer 442), for example a relatively stiff, closed cell foam. The second layer 442 can be a softer material (e.g., eggshell-type or other soft foam, cushion material, padding material, etc.). With this optional construction, the first layer 440 provides the core 410 with sufficient rigidity to maintain its structural shape when subjected to expected forces (e.g., when applied over the limb of a thrashing subject, the core 410 will permit only minimal, if any, bending along a length of the core 410). The second layer 442 is arranged to face the interior region 422 (FIG. 16B), and serves to minimize trauma to the subject's body. In other embodiments, the core 410 can have a singular construction (e.g., a single layer or material); in other embodiments, the core 410 can include three or more different materials (e.g., a strip(s) of plastic can be inserted between the first and second layers 440, 442 to increase a longitudinal rigidity of the core 410). While the core 410 has been generally shown as a continuous body, in other embodiments one or more cutouts can be formed through a wall thickness of the core 410 that enhance heat transfer from the subject's body during use (thus effectively serving as a cooling mechanism).

Returning to FIGS. 16A and 16B, the liner 412 can be akin to a sleeve, sized and shaped to cover the core 410 (as a point of reference, the liner 412 is shown as not extending about the opposing edges 430, 432 for ease of illustration). In some embodiments, the liner 412 is formed of a lightweight, waterproof material (e.g., nylon) appropriate for contacting human skin (e.g., providing infection control). In related embodiments, the core 410 can be removably inserted into the liner 412 (e.g., a zipper, elastic, or similar closure device can be provided at one or both ends of the liner 412); with these and similar constructions, the liner 412 can be removed from the core 610 and washed between uses. Regardless, the liner 612 is sized and shaped to mimic the gap 434 (i.e., when placed over the core 410, the liner 412 forms an incomplete tube), thus permitting the opposing edges 430, 432 of the core 410 to be forced toward or away from one another as described above.

The flap 414 is formed of a durable, high strength, stretchable material (e.g., stretch-like or elastic fabric). As generally reflected by FIG. 16B, the flap 414 is attached to (or formed by) an exterior surface of the liner 412 (e.g., proximate the first edge 430 upon final assembly of the liner 412 over the core 410) and extends to a free end 450. The flap 414 can be pulled and manipulated to impart a force onto the liner 412 than in turn is transferred onto the core 410, causing the core 410 to assume a smaller size or diameter as described above (i.e., bringing the opposing edges 430, 432 toward one another). Further, the flap 414 and/or the liner 412 carry or form one or more fastening mechanisms for securing the flap 414 to the liner 412 in the tightened or contracted arrangement. For example, complementary fasteners or fastening materials (e.g., hook-and-loop type complementary fastener materials such as Velcro®) can be provided. In one non-limiting example, a strip 452 of book or loop material is formed or carried on an underside of the flap 414. First and second strips 454, 456 of complementary hook or loop material (i.e., complementary to the hook or loop format of the strip 452 carried by the flap 414) are formed or carried by an exterior face of the liner 412 at discrete locations appropriate for interfacing with the flap strip 452 during use. In some examples, materials of the first and second strips 454, 456 can differ from one another, with a construction of the first strip 454 selected to achieve a lighter hold with the flap strip 452 as compared to a construction of the second strip 454.

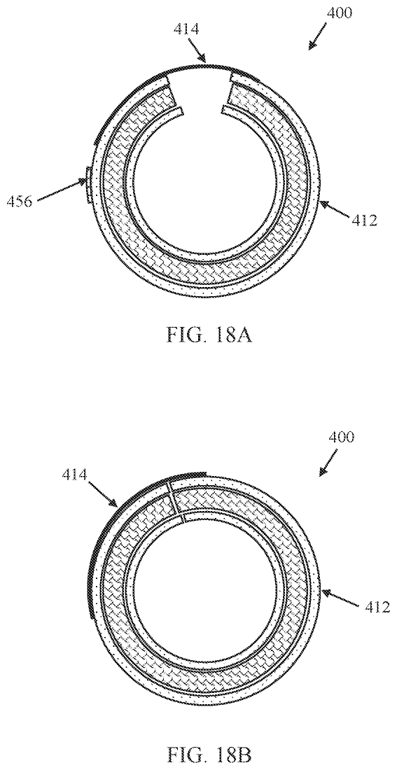

The optional configuration of the complementary fastening materials 452-456 can facilitate quick application and securement of the limb restraining device 400 to a subject's limb. For example, immediately upon placement of the subject's limb within the interior region 422, the free end 450 of the flap 414 can be directed toward the liner 412, with the flap strip 452 contacting and engaging with the first strip 454 as generally reflect by FIG. 18A. In this initial arrangement, the limb restraining device 400 may not be snug about the subject's limb, but is generally held in place via the interface between the flap strip 452 (FIG. 16B) and the first strip 454 (FIG. 16B). When ready, the user can then readily disengage the flap 414 from the liner 412 and apply a pulling force. This action, in tum, causes the limb restraining device 400 to contract as generally shown in FIG. 18B. Once the user is satisfied with a fit of the contracted limb retraining device 400 to the subject's limb, the flap 414 can again be directed toward the liner 412, bringing the flap strip 452 into engagement with the second strip 456. The high hold strength between the flap strip 452 and the second strip 456 ensures that the limb retraining device 400 remains snugly secured over the subject's limb. Alternatively, other devices or mechanisms can be used to secure the limb restraining device 400 that may or may not include complementary fastening materials.

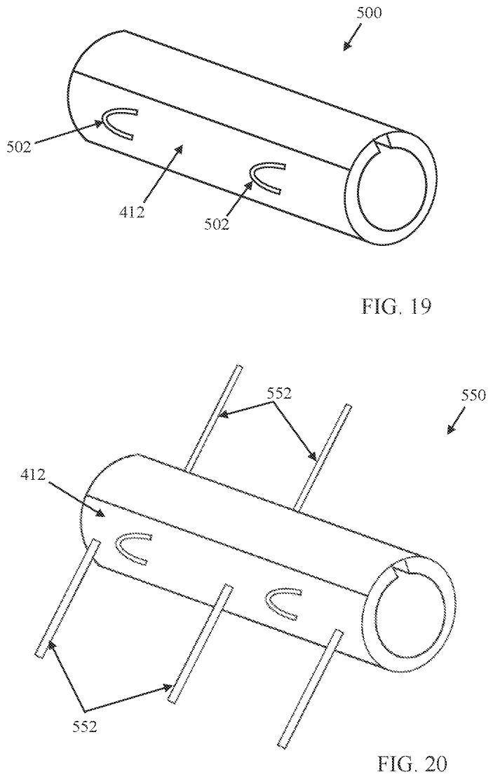

Returning to FIGS. 16A and 16B, the limb restraining device 400 can optionally include or carry one or more additional features that further promote use and/or subject restraint. For example, another limb restraining device 500 is shown in FIG. 19. The limb restraining device 500 can be highly akin to the limb restraining device 400 described above, and further includes one or more external handles 502. The handle(s) 502 can be secured to or formed by the liner 412, and can have a soft foam covering. Where provided, the handle(s) 502 provide a convenient surface for grasping by a user, for example when securing a subject's limb, using the limb restraining device 500 as a self-defense item or shield, using the limb restraining device 500 to direct a subject to a corner or wall, etc.

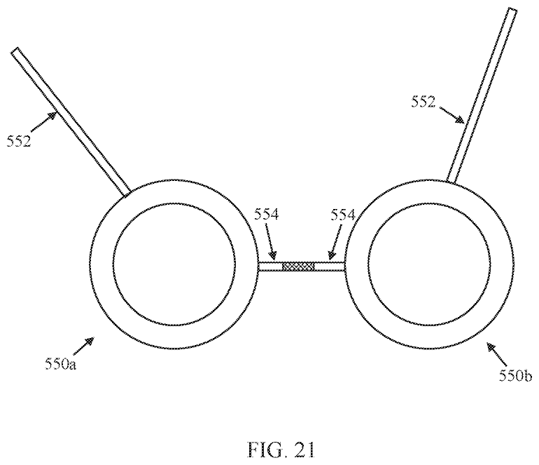

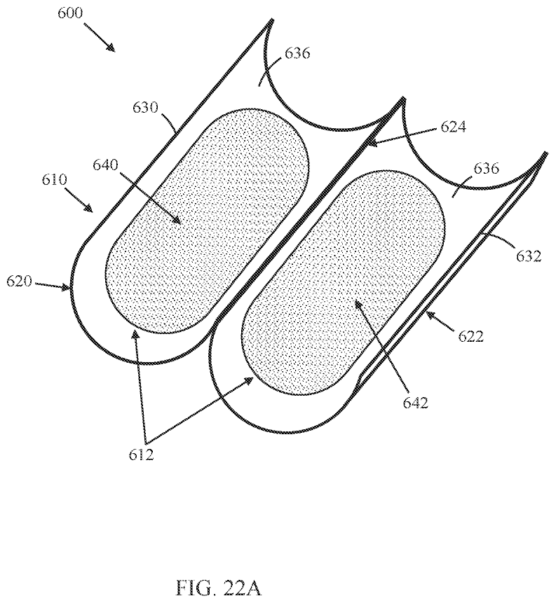

In other embodiments, one or more straps can be provided with the limb restraining devices of the present disclosure. For example, another limb restraining device 550 is shown in FIG. 20. The limb restraining device 550 can be highly akin to the limb restraining devices 400, 500 described above, and further includes one or more straps 552. The strap(s) 552 are secured to and extend from an exterior of the liner 412 and can include a quick-release buckle (not shown). With this configuration, the straps 552 can be coupled to another object such as a firm backboard, ambulance gurney, ER gurney, bed, etc. In related embodiments, and with additional reference to FIG. 21, straps of differing lengths can be provided. In the embodiment reflected by FIG. 21, two of the limb restraining devices 550a, 550b are generally illustrated, each having at least one shorter length strap 554 (along with one more of the longer length straps 552). With the first limb restraining device 550a applied to one of a subject's arms (or legs) and the second limb restraining device 550b applied to the other of the subject's arms (or legs), the shorter length straps 554 from the two limb restraining devices 550a, 550b can be coupled to one another to provide additional restraints on movement by the subject.

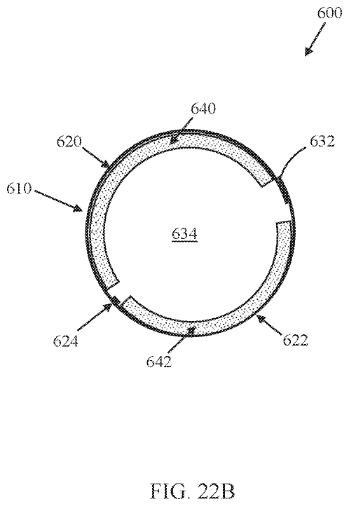

Another example of a limb restraining device 600 in accordance with principles of the present disclosure is shown in FIGS. 22A and 22B. The limb restraining device 600 includes a base 610, padding 612 (referenced generally), and an optional outer liner or sleeve (not shown). Details on the various components are provided below. In general terms, the base 610 has a clam shell-type configuration, and is sized and shaped for placement over and/or around a subject's limb. The padding 612 is secured to an interior surface of the base 610. Where provided, the liner covers the base 610 and the padding 612. The limb restraining device 600 can quickly be applied and secured over or around a subject's limb, and optionally includes or carries one or more additional features that assist in further restraining movement by the subject.

The base 610 can assume various forms, and in some embodiments include first and second base sections 620, 622. The base sections 620, 622 can be identical or substantially identical, and each define a “C” or “U” (e.g., half circle) in transverse shape along a length thereof. By way of non-limiting example, the base sections 620, 622 can be formed by dividing an elongated tubular body lengthwise. A seam or hinge 624 connects an edge of the first base section 620 with an edge of the second base section 622. With this arrangement, the first base section 620 terminates at a free edge 630 opposite the seam 624, and the second base section 622 terminates at a free edge 632 opposite the seam 624. The seam 624 is configured to permit the base sections 620, 622 to articulate relative to one another (pivoting at the scam 624) such that the base 610 can readily be transitioned between an open state (FIG. 22A) and a closed state (FIG. 22B) in which the base 610 defines a closed interior region 634. In some embodiments, the base sections 620, 622 are sized and shaped such that the free edge 632 of the second base section 622 exteriorly overlaps the first base section 620 in the closed state, with a stiffness or similar property of the sections 620, 622 being sufficient to provide a locking effect in the closed state. In other embodiments, a latch (or similar mechanism), strap, etc., can be provided with the limb restraining device 600 to releasably hold or lock the base 610 in the closed state. Regardless, a subject's limb can be placed into one of the base sections 620, 622 in the open state; by then transitioning the base 610 to the closed state, the subject's limb will be secured within the interior region 634. With embodiments in which the base 610 is configured such that the free edge 632 of the second base section 622 exteriorly overlaps the first base section 620 in the closed state, the extent of overlap can be selected to best-size the interior region 634 relative to a size of the subject's limb being constrained.

The base sections 620, 622 can be formed of a strong, lightweight, relatively thin material such as hardened cardboard, plastic, etc. In some embodiments, a material of the base sections 620, 622 is recyclable. The seam 624 can be continuous along a length of the base sections 620, 622 (e.g., a continuous strip of flexible tape, cloth or similar material) and/or can include one or more hinges or similar mechanisms,

The padding 612 is provided on an interior face 636 of each of the base sections 620, 622 and can assume various forms appropriate for complaint interface with a subject's limb (e.g., soft foam, egg crate-type foam, cloth, etc.). In some embodiments, the padding 612 includes at least a first pad 640 provided with the first base section 620 and a second pad 642 provided with the second base section 622. With these and related embodiments, the pads 640, 642 are sized, shaped and located to not interfere with free pivoting movement of the base sections 620, 622 at the seam 624. The pads 640, 642 can be secured to the corresponding base section 620, 622 in various manners. In some examples, the limb restraining device 610 is configured such that the pads 640, 642 are removably associated with the corresponding base section 620, 622. For example, complementary fasteners or fastening materials (e.g., hook-and-loop type complementary fastener materials such as Velcro®) can be provided along the interior face 636 of each of the base sections 620, 622 and with the pads 640, 642. In related embodiments, a number of differently-configured pads can be available to a user (e.g., differing in terms of one or more of thickness, size, and shape); a user can then select the pads best suited for a particular subject and connect the selected pads to the base sections 620, 622 via the complementary fasteners. In other embodiments, the pads 640, 642 can be more permanently affixed to the corresponding base sections 620, 622 (e.g., adhesive, weld, etc.). In yet other embodiments, the padding 612 can consist of a single pad body extending along both of the base sections 620, 622.

Where provided, the outer liner or sleeve can be akin to the liner 412 described above with respect to the embodiment of FIGS. 16A and 16B. In general terms, the outer liner or sleeve is sized and shaped to cover the base 610 and the padding 612, and can be formed of a lightweight, waterproof material (e.g., nylon) appropriate for contacting human skin (e.g., providing infection control). The outer liner or sleeve can be configured to removably receive the base 610 and the padding 612 (e.g., a zipper, elastic, or similar closure device can be provided at one or both ends of the outer liner sleeve); with these and similar constructions, the outer liner can be removed from the base 610 and washed between uses. Regardless, the outer liner is sized and shaped to permit the base 610 to readily transition between the open and closed states.

The limb restraining device 600 can optionally include any of the additional features described above. For example, one or more external handles can be provided that are akin to the handles 502 (FIG. 19) described above. The handle(s) can be secured to or formed by the outer liner or the base 610 (with embodiments in which the outer liner is not included), and can have a soft foam covering. The optional handle(s) provide a convenient surface for grasping by a user, for example when securing a subject's limb, using the limb restraining device 600 as a self-defense item or shield, using the limb restraining device 600 to direct a subject to a comer or wall, etc. Additionally or alternatively, one or more straps can be provided with the limb restraining device 600. The strap(s) can be akin to the straps 552 (FIG. 20) described above, secured to and extend from an exterior of the outer liner or the base 610 (with embodiments in which the outer liner is not included) and can include a quick-release buckle. With this configuration, the straps can be coupled to another object such as a firm backboard, ambulance gurney, ER gurney, bed, etc. In related embodiments, and as described above with respect to FIG. 21, straps of differing lengths can be provided. In particular, shorter length straps provided with two of the limb restraining devices 600 can be coupled to one another to provide additional restraints on movement by the subject.

Regardless of an exact design, any of the limb restraining devices of the present disclosure are useful for a wide range of applications. For example, during the often dangerous and violent take-down phase of subduing a patient, suspect, prisoner, active shooter, and general assailants in healthcare settings, schools, malls, police arrests, public spaces, etc. The limb restraining devices can also be used, for example, by parent/caregiver teams at home.

In some embodiments of the present disclosure, a restraining system 700 that includes an upper torso restraining device 710, limb restraining devices 720 (any of the limb restraining devices described above), limb restraining devices 722 (any of the limb restraining devices described above that include straps), and optional cuff-type limb restraining devices 724. Regardless of an exact design, the system 700 can be used during the takedown phase in a rapid, safe, stepwise process. In some embodiments, the limb restraining device(s) can be contained in flat pouches on both sides of the upper torso restraining device to provide an all-in-one system for subduing violent individuals. The pouches could also contain one or more of sedative injection packs, first aid supplies, a cell phone, automatic alarms and site locators to draw other nursing/staff/security backup. In related embodiments, the system 700 can be configured such that were it removed from storage in a school hallway, patient room, hospital room, waiting room, etc., a locator beacon would automatically send an alert to the security office, 911, etc. Once pinned to the floor and subdued with sufficient immobilization via the upper torso restraining device 710, the limb restraining devices 720, 722 can be rapidly applied onto the subject's knees and elbows for transfer to a gurney. Wrist restraints with quick release plastic buckles can provide redundancy for the joint immobilizers.

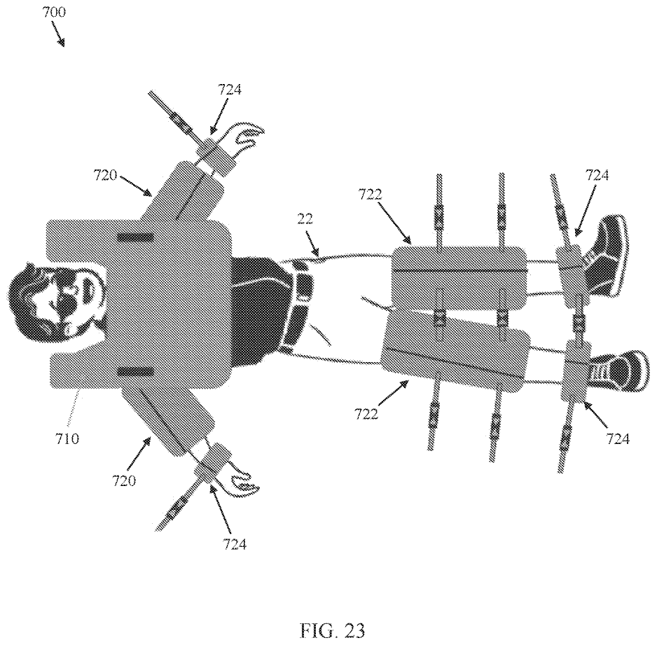

Once the subject 22 is subdued by professionals to a firm surface using the shell-type restraining devices (typically by two or more professionals, each responsible for one of the shell-type restraining devices), limbs of the subject 22 can be released from the corresponding shell-type restraining device, one limb at a time, and placed into a limb restraining device (e.g., any of the limb restraining devices described above) as part of a contain and transfer system. As a point of reference, while FIG. 23 illustrates the subject's legs being secured to one another via straps provided with limb restraining devices 722, in other embodiments the limb restraining devices applied to the subject's arms (i.e., the limb restraining device 720 in FIG. 23) can also include straps, such as short straps that can be coupled to one another to thereby secure the subject's arms in a handcuff-type arrangement. The limb restraining devices can then be strapped or “clicked” together as described above. The subject 22, with the limb restraining devices applied, can then be safely moved or transferred, for example rolled onto a backboard and transferred to a bed with more permanent restraints and/or released once calm.

The systems and devices of the present disclosure provide a marked improvement over previous designs. The upper torso and other shell-type restraining devices and the limb restraining devices can be used alone or in combination. Regardless, the systems and devices of the present disclosure can solve, for example, the problem of approaching violent/agitated youth and adults (as a last resort when de-escalation techniques are not effective and the risk is too high to leave the subject un-restrained) in a way that protects the safety and dignity of the subject while protecting the staff subduing the subject. The upper torso restraining device can be used as a shield and to quickly pin the subject to the floor or other flat surface. The upper torso restraining device and the limb restraining device(s) can essentially provide a “hands on, hands off” approach for quickly restraining the subject by a team. One or more of the upper torso restraining devices could be mounted to walls of a hospital, school, classroom, mall, workplace, etc. (similar to AEDs), with the general public trained how to use them, but particularly professionals such as emergency responders, police, security, nurses, doctors, orderlies, group home staff, emergency room staff, etc. The limb restraining devices can be easily folded to a very small size and could similarly be mounted to walls of a hospital, school, classroom, mall, workplace, etc. (similar to AEDs), with the general public trained how to use them, but particularly trained teams of two or more professionals such as emergency responders, police, security, nurses, doctors, orderlies, group home staff, emergency room staff, etc.

In some examples, the systems of the present disclosure can be referred to as a Safeguard Sheild and Containment System (SSCS) that consists of two elements: a Safeguard Containment Device (SCD) and four Safeguard Limb Devices (SLD). Both devices are padded and extremely lightweight, designed for safe use with both adult and pediatric patients. In some examples, central to the SSC System is the SCD which shields staff from kicks, punches, and spittle; and serves to contain the torso, hips, and/or limbs against a flat surface, allows visualization of the patient's face and neck, and minimizes contact between staff and patient. The SCD can be foldable for wall storage, mechanically size-adjustable, well-padded, and extremely lightweight to prevent use for harms. Optional handles, quick release straps, and a kneeling surface allow staff to secure the SCD to the floor, wall, or ER bed while the underside of the SCD curves shell-like over the patient's chest and neck preventing suffocation from body weight during containment events. The SLD can be cylindrical in shape, padded inside and out, and ranges approximately 16-22 inches long for use with an average adult (larger sizes are available for very large adults and smaller sizes are available for pediatric populations). Using its optional two handles and other novel features, team members can use the SLD in multiple ways, tailorable to each situation. Each SLD can be employed as a lightweight shield for staff during the approach, firm structure for maneuvering an active patient, and rapid immobilization of limbs in the extended position. The SLD's provide flexible use, adaptable by emergency response teams for optimizing their containment process, with recyclable options to provide environmentally responsible single-patient use yet washable for disinfection and repeated use if desired. Similar to a Formula 1 pit stop, the SSC System devices in the hands of a trained response team can transform the dangerous, prolonged melee of unprotected engagement with combative patients into a rapid, systematic process of safe containment in thirty seconds or less. The speed of containment and multi-functional devices are designed to dramatically improve safety and the optics of containment events; reduce rates of injury and risk of liability; and stem the tide of nurse attrition due to fear, trauma, and disabling injury.

Although the present disclosure has been described with reference to preferred embodiments, workers skilled in the art will recognize that changes can be made in form and detail without departing from the spirit and scope of the present disclosure.

1.-32. (canceled)

33. A restraining system comprising:
a first limb restraining device including:
a base comprising first and second base sections each defining a C-like transverse shape,
wherein adjacent edges of the first and second base sections are hingedly secured to one another at a seam,
padding carried along an interior face of the first and second base sections,
wherein the first limb restraining device is transitionable between an open state and a closed state, and further wherein the closed state includes the base defining an enclosed interior region for receiving and maintaining a portion of a limb of a human adult subject.

34. The restraining system of claim 33, wherein the first limb restraining device is configured to be applied about a joint of the subject.

35. The restraining system of claim 33, wherein the base has a clam shell-type configuration.

36. The restraining system of claim 33, wherein the first limb restraining device further includes at least one handle extending outwardly from the base.

37. The restraining system of claim 36, wherein the first limb restraining device is configured for use as a shield by user a grasping the at least one handle.

38. The restraining system of claim 33, wherein the first limb restraining device further includes at least one strap.

39. The restraining system of claim 38, further comprising a second limb restraining device configured to be selectively connected to the first restraining device by the at least one strap.

40. The restraining system of claim 33, wherein the padding include a first pad releasably secured to the first base section and a second pad releasably secured to the second base section.

41. The restraining system of claim 33, further comprising:
a shield device including a platform and a plurality of pillars extending from the platform, the platform and pillars collectively defining an interior region;
wherein the shield device is configured to pin at least a body part of the human adult subject to a surface, including the body part residing in the interior region.

42. The restraining system of claim 41, wherein:
the platform defines a leading end opposite a trailing end, and a first side opposite a second side, the plurality of pillars includes:
a first pillar extending from the platform proximate the first side,
a second pillar extending from the platform proximate the first side and spaced from the first pillar,
a third pillar extending from the platform proximate the second side,
a fourth pillar extending from the platform proximate the second side and spaced from the first pillar,
wherein the first, second, third and fourth pillars each terminate at an end, and the ends collectively define a lower face of the shield device.

43. The restraining system of claim 42, wherein the shield device further includes:
a first support surface extending between the first and second pillars;
a second support surface extending between the third and fourth pillars;
wherein a distance between the lower face and the platform is greater than a distance between the lower face and the first support surface, and is greater than a distance between the lower face and the second support surface.

44. The restraining system of claim 43, wherein the shield device is configured to provide a restraining state relative to the subject, including a chest of the subject below and spaced from the platform, and at least one of a first shoulder and a first arm of the subject pinned to the surface by the first support surface.

45. The restraining system of claim 44, wherein the restraining state includes the platform arranged over and spaced from a neck of the subject.

46. The restraining system of claim 43, wherein the shield device is configured to provide a restraining state relative to the subject, including a limb of the subject pinned to the surface by at least one of the first support surface and the second support surface.

47. The restraining system of claim 41, wherein the plurality of pillars are configured to support the platform relative to the surface under conditions where a weight is placed onto the platform.

48. The restraining system of claim 41, wherein the interior region is sized and shaped for placement over an upper torso, neck and at least a portion of a head of the subject.

49. The restraining system of claim 41, wherein each of the plurality of pillars are transitionally connected to the platform such that shield device is transitionable between a deployed state and a storage state.
